(19) United States
(12) Patent Application Publication
(10) Pub. No.: US 2024/0138973 A1
(43) Pub. Date: May 2, 2024

(54) IN-SITU FENESTRATION DEVICES WITH CUTTING STAPLES

(71) Applicant: Medtronic Vascular, Inc., Santa Rosa, CA (US)

(72) Inventors: Julie-Anne McBARRON, Galway (IE); Tomas P. KITT, Galway (IE); Lara M. FRAMPTON, Galway (IE); Patrick KING, Galway (IE)

(21) Appl. No.: 18/384,409

(22) Filed: Oct. 27, 2023

Related U.S. Application Data
(60) Provisional application No. 63/420,223, filed on Oct. 28, 2022.

(51) Int. Cl. A61F 2/07

(52) U.S. Cl. CPC A61F 2/07; A61F 2002/075; A61F 2220/0066

(57) ABSTRACT

An in-situ fenestration device including a cutting staple, a delivery staple, and a backplate catheter. The cutting staple has a delivery position, a cutting position, and a deployed position. The delivery catheter is configured to deliver the cutting staple to an in-situ fenestration site of a graft material situated within an artery. The backplate catheter is fixated to the delivery catheter as the cutting staple transitions from the delivery position to the cutting position to cut an in-situ fenestration at the in-situ fenestration site. The backplate catheter includes one or more channels configured to transition the cutting staple from the cutting position to the deployed position anchoring the cutting staple to the graft material around a periphery of the in-situ fenestration.

### CROSS-REFERENCE TO RELATED APPLICATION

This application claims the benefit of U.S. provisional application Ser. No. 63/420,223 filed on Oct. 28, 2022, the disclosure of which is hereby incorporated in its entirety by reference.

### TECHNICAL FIELD

The present disclosure relates to in-situ fenestration devices with cutting staples.

### BACKGROUND

In-situ fenestration (ISF) has seen limited applicability to aortic stent grafts for endovascular aneurysm repair (EVAR) and thoracic endovascular aneurysm repair (TEVAR). In-situ fenestration of aortic stent grafts can be used to maintain perfusion to blood vessels (e.g., aortic side branch arteries or peripheral arteries) located in an area excluded by a stent graft. In-situ fenestration may be used to fenestrate (e.g., create a new opening or hole) in a stent graft in-situ (e.g., in the place of the stent graft) following deployment of the stent graft within a vascular system. Application of ISF has been typically limited to removing unintentional coverage of blood vessels (e.g., arteries) upon deployment of a stent graft, but has rarely been used in elective scenarios.

### SUMMARY

In one embodiment, an in-situ fenestration device including a cutting staple, a delivery staple, and a backplate catheter is disclosed. The cutting staple has a delivery position, a cutting position, and a deployed position. The delivery catheter is configured to deliver the cutting staple to an in-situ fenestration site of a graft material situated within an artery. The backplate catheter is fixated to the delivery catheter as the cutting staple transitions from the delivery position to the cutting position to cut an in-situ fenestration at the in-situ fenestration site. The backplate catheter includes one or more channels configured to transition the cutting staple from the cutting position to the deployed position anchoring the cutting staple to the graft material around a periphery of the in-situ fenestration.

In another embodiment, an in-situ fenestration cutting staple is disclosed. The in-situ fenestration cutting staple includes a proximal base, a leading edge, a medial portion extending from the proximal base toward the leading edge, and a cutting edge outwardly tapering from the leading edge toward the medial portion. The cutting edge is configured to cut into a graft material at an in-situ fenestration site to form an in-situ fenestration.

In yet another embodiment, a method of forming an in-situ fenestration is disclosed. The method includes advancing a cutting staple within a delivery catheter where the cutting staple include a cutting edge. The method also includes further advancing the cutting staple such that the cutting edge cuts into graft material at an in-situ fenestration site to form an in-situ fenestration. The method also includes yet further advancing the cutting staple such that the cutting staple transitions into an anchored position in which the cutting staple pinches the graft material at a periphery of the in-situ fenestration.

### BRIEF DESCRIPTION OF THE DRAWINGS

FIG. 1A depicts a partial cut away, schematic, side view of an abdominal aorta and right and left renal arteries extending therefrom where a stent graft excludes the right and left renal arteries from blood perfusion.

FIG. 1B depicts a partial cut away, schematic, side view of an aortic arch branching into a brachiocephalic artery, a left common carotid artery, and a left subclavian artery where a stent graft excludes the left subclavian artery from blood perfusion.

FIG. 2A depicts an isolated, perspective view of a circular cutting staple in a cutting position.

FIG. 2B depicts a perspective view of a circular cutting staple in a deformed position within a fenestration in a stent material.

FIG. 3A depicts a partial cut away, schematic, side view of an aortic arch and a branch artery (e.g., the left subclavian artery) thereof.

FIG. 3B depicts a partial cut away, schematic, side view of a backplate catheter including a backplate carried at a distal end thereof as part of an in-situ fenestration system according to one or more embodiments.

FIG. 3C depicts magnified area 3C of FIG. 3B.

FIG. 3D depicts a partial cut away, schematic, side view of the backplate catheter advanced through a branch artery such that the backplate presses against the graft material of a stent graft.

FIG. 3E depicts a partial cut away, schematic, side view of a steerable catheter having a distal end facing the stent graft and the circular cutting staple advancing through the steerable catheter.

FIG. 3F depicts a partial cut away, schematic, side view of the steerable catheter fixating to the backplate catheter.

FIG. 3G depicts a partial cut away, schematic, side view of a piston advancing through the steerable catheter to advance the circular cutting staple so that the cutting edge cuts (e.g., shears) graft material of the stent graft at the fenestration site.

FIG. 3H depicts a partial cut away, schematic, side view of the circular cutting staple in the deformed state, thereby forming a pinch point between the cutting edge and the proximal base of the circular cutting staple.

FIG. 3I depicts a partial cut away, schematic, side view of the backplate catheter and the steerable catheter retracting through the branch artery and the aortic arch, respectively, and away from the deployed circular cutting staple having deformed fingers.

FIG. 4 depicts a schematic, perspective view of the circular cutting staple in a deployed position around the fenestration.

FIG. 5A depicts an isolated, perspective view of a circular cutting stent in a cutting position.

FIG. 5B depicts an isolated, perspective view of the circular cutting stent in a deployed position.

FIG. 6A depicts a partial cut away, schematic, side view of an aortic arch and a branch artery (e.g., the left subclavian artery) extending therefrom.

FIG. 6B depicts a partial cut away, schematic, side view of a steerable catheter having a distal end facing a stent graft.

FIG. 6C depicts a partial cut away, schematic, side view of a rotatable coil advancing via rotation through the stent graft material of the stent graft and a backplate of a backplate catheter.

FIG. 6D depicts a fragmented, schematic view of the steerable catheter, the backplate catheter, and the rotatable coil extending into the backplate catheter from the steerable catheter.

FIG. 6E depicts a fragmented, schematic view of graft material of the stent graft with the rotatable coil rotating therethrough.

FIG. 6F depicts a partial cut away, schematic, side view of the rotatable being pulled linearly in a proximal directed as depicts by the downward arrow to pull the distal ends of the steerable catheter and the backplate catheter, respectively, together and fix the position of the steerable catheter and the backplate relative to each other.

FIG. 6G depicts a partial cut away, schematic, side view of the circular cutting stent in a cutting position advancing as represented by arrows from the outer lumen so that the cutting edge cuts (e.g., shears) the graft material of the stent graft at the fenestration site.

FIG. 6H depicts a partial cut away, schematic, side view of the circular cutting stent in a partially deployed position where the distal end has taken on a preformed shape.

FIG. 6I depicts a partial cut away, schematic, side view of the rotatable coil rotating in a proximal direction to disengage the rotatable coil from the backplate, thereby disconnecting the steerable catheter from the backplate catheter.

FIG. 6J depicts a partial cut away, schematic, side view of the circular cutting stent in a deployed position where the proximal end has taken on a preformed shape.

FIGS. 6K and 6L depict a schematic, side view and a schematic, isolated view, respectively, of self-expanding region 204 before it has expanded.

FIGS. 6M and 6N depict a schematic, side view and a schematic, isolated view, respectively, of self-expanding region 204 after expansion.

FIG. 7 depicts a schematic, perspective view of the circular cutting stent in a deployed position around the fenestration.

FIG. 8A depicts a perspective, schematic view of the fenestration needle configured to create a fenestration in a deployed stent graft.

FIG. 8B depicts a perspective, schematic view of a grommet deployed within a fenestration.

FIG. 8C depicts a perspective, isolated, schematic view of the grommet.

FIG. 9A depicts an isolated, schematic view of an in-situ fenestration system including a sheath advanced through deployed aortic stent graft to a renal artery location.

FIG. 9B depicts a perspective, schematic view of the sheath tracked over a guidewire to the renal artery location.

FIG. 10A depicts a perspective, schematic view of the fenestration needle advancing through an initial puncture in the stent graft to increase the size of the fenestration.

FIG. 10B depicts a perspective, schematic view of the fenestration needle where a balloon radially expands discrete needles of the fenestration needle to displace the graft material of the stent graft to further increase the size of the fenestration.

FIGS. 10C and 10D depict a cross-section, side view and front view, respectively, of the fenestration needle in a delivery position.

FIGS. 10E and 10F depict a cross-section, side view and front view, respectively, of the fenestration needle in a deployed position.

FIGS. 11A through 11D depict cut away schematic, side views of operations of a grommet delivery system for reinforcing a fenestration with a grommet.

FIG. 12A depicts a schematic, plan view of a rotating member system including a rotatable force member.

FIG. 12B depicts a schematic, plan, perspective view of the rotating member system including the rotatable force member.

FIG. 12C depicts a schematic, perspective view of the rotating member system with the rotatable force member removed.

FIG. 12D depicts a perspective, isolated view of the rotatable force member.

FIG. 12E depicts a top, isolated view of the rotatable force member including a rotation wire within a channel.

FIG. 13A depicts a perspective, schematic view of the grommet including first and second sections.

FIG. 13B depicts a perspective, schematic view of the grommet including first and second sections where the second section includes flanges separated by slits.

FIG. 14A depicts a perspective, schematic view of the rotatable force member proximal to the lumen of the grommet and in the unrotated position.

FIG. 14B depicts a perspective, schematic view of the rotatable force after it advanced through the lumen of the grommet and transitions from the unrotated position to the rotated position.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” are positions distant from or in a direction away from the clinician, and “proximal” and “proximally” are positions near or in a direction toward the clinician. For the stent-graft prosthesis, “proximal” is the portion nearer the heart by way of blood flow path while “distal” is the portion of the stent-graft further from the heart by way of blood flow path.

The following detailed description is merely exemplary in nature and is not intended to limit the invention or the application and uses of the invention. Although the description is in the context of treatment of blood vessels such as the aorta, coronary, carotid, and renal arteries, the invention may also be used in any other body passageways (e.g., aortic valves, heart ventricles, and heart walls) where it is deemed useful.

In-situ fenestration (ISF) has seen limited applicability to aortic stent grafts for endovascular aneurysm repair (EVAR) and thoracic endovascular aneurysm repair (TEVAR). In-situ fenestration of aortic stent grafts can be used to maintain perfusion to blood vessels (e.g., aortic side branch arteries or peripheral arteries) located in an area excluded by a stent graft. In-situ fenestration may be used to fenestrate (e.g., create a new opening or hole) in a stent graft in-situ (e.g., in the place of the stent graft) following deployment of the stent graft within a vascular system. Application of ISF has been typically limited to removing unintentional coverage of blood vessels (e.g., arteries) upon deployment of a stent graft, but has rarely been used in elective scenarios.

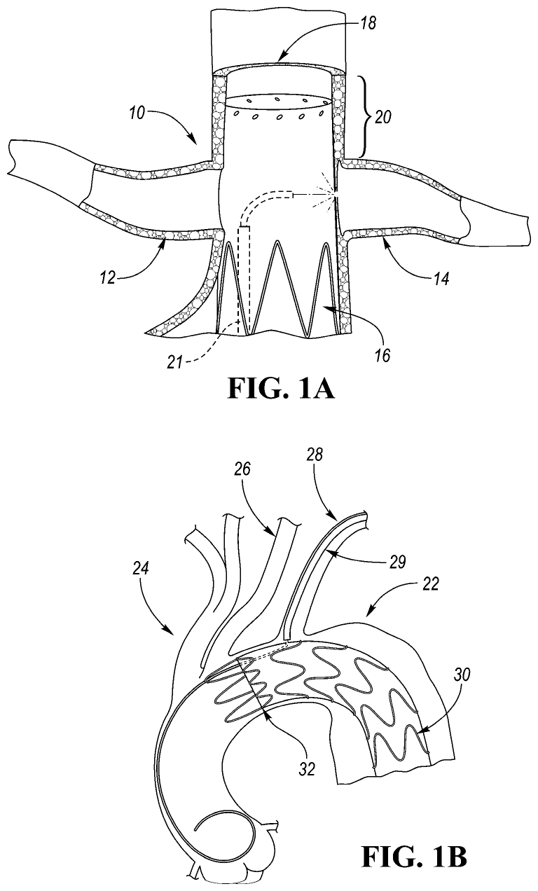

FIG. 1A depicts a partially cut away, schematic, side view of abdominal aorta 10 and right renal artery 12 and left renal artery 14 extending from abdominal aorta 10. Right and left renal arteries 12 and 14 may be referred to generally as the renal arteries. Stent graft 16 includes proximal end 18 and a distal end (not shown). Proximal end 18 of stent graft 16 lands in landing zone 20 of abdominal aorta 10. Stent graft 16 extends from landing zone 20 to exclude perfusion to right renal artery 12 and left renal artery 14. An in-situ fenestration at the exclusion areas (e.g., using laser fenestration device 21) can be used to perfuse right renal artery 12 and left renal artery 14. Perfusion may result from blood flow through the fenestration alone or through a branch stent graft inserted into the fenestration after it is created and extending into the branch artery.

FIG. 1B depicts a partial cut away, schematic, side view of aortic arch 22 branching into brachiocephalic artery 24, left common carotid artery 26, and left subclavian artery 28. Brachiocephalic artery 24, left common carotid artery 26, and left subclavian artery 28 may be referred to generally as side branch arteries. Stent graft 30 includes proximal end 32 and a distal end (not shown). Stent graft 30 extends to exclude perfusion to left subclavian artery 28. An in-situ fenestration (e.g., using laser fenestration device 29) at the exclusion area created at left subclavian artery 28 can be used to perfuse left subclavian artery 28 (e.g., via the fenestration or a later-deployed branch stent graft).

In-situ fenestration may provide a solution for implementing stent grafts with patients having hostile neck anatomy within their abdominal aorta. Current stent graft seal technology is unsuitable for many aortic anatomies. Many aortic abdominal and thoracic aortic aneurysms present either a relatively short seal zone (e.g., 0 to 10 millimeters) and/or a high degree of landing zone angulation. Examples of such anatomies include a short neck aneurysm, no neck thoraco-abdominal aneurysm, reverse conical neck, and highly angled aneurysm neck with a short landing zone inner curve. Under these circumstances, an alternative landing zone may be used that excludes perfusion to peripheral arteries (e.g., the renal arteries). In-situ fenestration may be used to open these excluded areas to permit blood perfusion. However, adequate in-situ fenestration processes and related devices/systems have not been proposed to realize the potential of in-situ fenestration in this regard.

Accordingly, clinicians (e.g., doctors or physicians) have investigated other techniques for modifying stent grafts for EVAR and TEVAR patients. The existing techniques (e.g., dedicated off-the-shelf multibranch devices, custom-made multibranch devices, clinician modified devices, and peripheral techniques) do not adequately modify stents grafts to completely address blood perfusion.

For instance, dedicated off-the-shelf multibranch devices may have low patient applicability due to variability in the anatomy of patients. The geometry to accommodate multiple branches on a dedicated branch device can be complicated to determine. Procedures to deploy these devices are complex. Branching canulation and/or stenting can be complicated because the devices are susceptible to rotational or axial misalignment.

An alternative technology is a custom-made multibranch device. However, these devices require a significant lead time (e.g., 6 to 8 weeks) and are not available for emergent cases. Moreover, custom ordered devices may still be susceptible to axial and rotational misalignment.

Clinicians have modified stent grafts themselves before deploying the stent graft in the vascular system of the patient. Physicians can partially deploy an off-the-shelf stent graft on a sterile field and make fenestrations based on patient specific anatomy. This type of “back table” modification of an off-the-shelf stent graft may have one or more benefits. Eye cautery (e.g., thermal energy) may be used to clean and/or seal any frayed and/or cut fiber ends at the fenestration boundary. The size of the fenestration is customizable without post dilation, which may cause material damage. The fenestrations can be made using three-dimensional (3D) reconstructions from patient specific computed tomography (CT) scans. The fenestrations can be reinforced with sutures and/or guidewires to make a durable interface between the main stent graft and the branch stent graft. However, these procedures include unloading of the stent graft so that it can be modified with a fenestration. Reloading the stent graft is a challenge due to the low profile and high packing density of the stent graft in the radially compressed, delivery state. These modifications are typically labor and time intensive.

Techniques for providing blood flow to peripheral blood vessels used in connection with off-the-shelf stent grafts have also been proposed. Clinicians can deploy off-the shelf stent grafts in parallel with these techniques to permit blood perfusion to peripheral arteries and respective organs. Examples of these types of technologies chimneys, snorkels, and sandwich techniques. A chimney structure may be applied in the abdominal aorta and may include a renal chimney and a seal zone distal to a lower chimney. A different structure may be applied in the aortic arch where blood flows into a chimney from the aortic arch and blood flows out of the chimney into the left common carotid artery, and blood flows into a periscope from the aortic arch and blood flows out of the periscope into the left subclavian artery. Another technique is referred to as a sandwich. Blood flows into the celiac artery and superior mesenteric artery (SMA) from sandwich parallel chimneys. These techniques may have one or more of the following benefits: (1) available for emergent cases; (2) configurations can be adapted for patient-specific anatomies (e.g., ballerina techniques); and/or (3) planning using 3D reconstructions from patient specific CT scans. However, these techniques have durability concerns and potential mid or long-term occlusion risks relating to challenging hemodynamics.

Due to one or more drawbacks of the existing technologies identified above, there has been interest in developing in-situ fenestration technology that addresses one or more of the drawbacks identified above. In-situ fenestration encompasses processes in which apertures are made in a fully or partially deployed stent graft inside of a patient. Under limited circumstances, in-situ fenestration has been employed to provide perfusion in the aortic arch, the visceral segment, and the iliac arteries. In the aortic arch, in-situ fenestration can be made in a retrograde direction (e.g., outside of the stent graft) using supra-aortic access. Other anatomies may use in situ fenestration using an antegrade technique (e.g., inside the stent graft). In-situ fenestration may have one or more of the following benefits: (1) provides a multibranch solution independent of patient anatomical constraints thus providing for a larger applicability; (2) can be performed using off-the-shelf stent grafts; and/or (3) may avoid time-consuming “back-table” modification and technically challenging reloading into delivery systems.

However, current in-situ fenestration techniques suffer from one or more drawbacks. Current in-situ fenestration methods result in relatively small size apertures where aggressive post-dilation is used to accommodate a branch stent graft. Needle in-situ fenestration uses a needle to create an initial fenestration. Laser fenestration uses a laser ablation catheter having a diameter of 2.0 to 2.5 millimeters. Radio frequency (RF) ablation may also be used. One example of an RF ablation method uses a 0.035 inch powered wire. As a drawback, damage to the graft material during fenestration expansion adds to procedural variability and makes durability testing difficult. Additionally, lack of standardized protocols results in lack of consistency in fenestrations, thereby inhibiting consistent anticipation of intermediate and long-term durability.

In one or more embodiments, in-situ fenestration process and/or related devices are disclosed that at least partially addresses one or more of the following drawbacks and/or at least partially provides one or more of the following benefits. A potential drawback of existing technology is anatomical variability limiting patient applicability of dedicated off-the shelf branch devices. A potential benefit of in-situ fenestration is customization of off the shelf stent grafts that is independent of anatomical constraints. Custom devices have been proposed but take a relatively long time (e.g., 6-8 weeks) for manufacture and deliver, and may not be available for emergent cases. A potential benefit of in-situ fenestration is application to off-the-shelf devices with no manufacturing or shipping delays.

Another potential drawback relates to “back table” modification of off-the-shelf devices by clinicians. These modified devices are difficult to reload, limiting adoption of this method. In-situ modification of a stent graft occurs in-situ, and thereby eliminating the step of reloading the device into a delivery system. Custom and “back table” modified devices are susceptible to axial or rotational misalignment which can impact vessel canulation. Fenestrations created in-situ after the deployment of a stent graft are independent of the position of the main graft.

Current in-situ fenestration procedure lack standardization in terms of initial fenestration source and post dilation procedures. A potential benefit of standardization would be the reduction or elimination of severe post dilation steps that can cause unpredictable damage to a graft material.

Current in-situ fenestration procedures may result in cut fibers and/or ripped material. These drawbacks may represent a source of procedural variability and may limit the long-term durability and seal of the fenestration and branch stent graft interface. One or more embodiments disclose a method for sealing cut fibers that help prevent continued breakdown of the fenestration and branch stent graft interface.

Current fenestration techniques start with a small initial fenestration that is aggressively post dilated to accommodate a branch graft which can result in the tearing of the graft material. Some graft materials use cutting balloons for post dilation, which may cause additional cut fibers and material damage. One or more embodiments disclose a method and/or device for forming a fenestration in-situ of a size and shape that involves little or no post dilation and/or cutting balloons.

Power sources (e.g., laser and RF ablation) for current in-situ fenestrations may create steam bubbles and generate char particles that can pose embolic risk. One or more embodiments disclose a method and/or device to allow in-situ fenestration creation while minimizing steam bubbles and char formation.

In one or more embodiments, an in-situ fenestration device for forming a fenestration in graft material of a deployed stent graft is disclosed. The in-situ fenestration device may include a cutting staple or stent. The cutting staple or stent may have a distal cutting edge configured to cut the graft material to form the fenestration. The cutting staple or stent may have a cutting position in which the graft material is cut and a deformed position in which the cutting staple or stent crimps the fenestrated graft material adjacent to the fenestration.

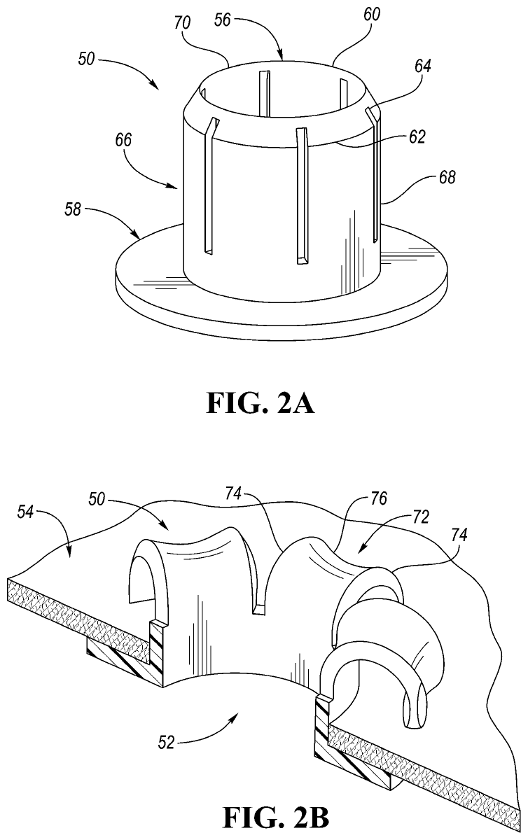

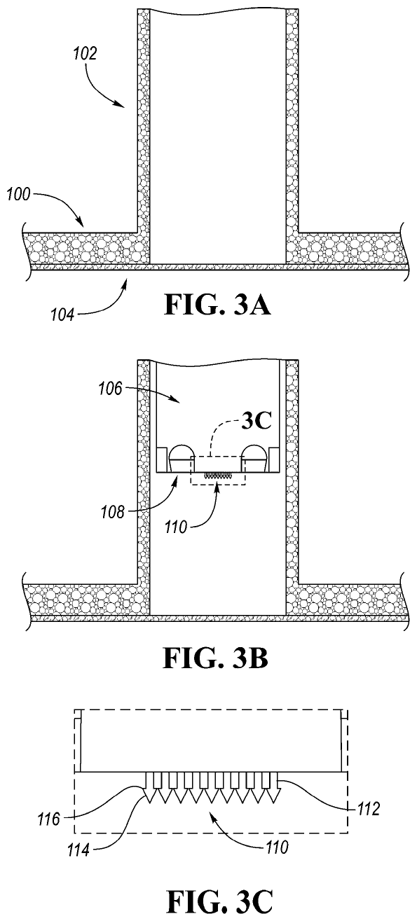

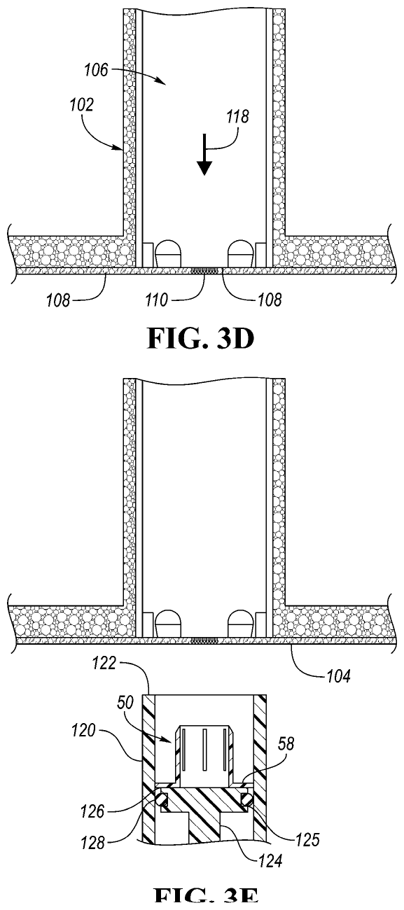

FIG. 2A depicts an isolated, perspective view of circular cutting staple 50 in a cutting position. In one or more embodiments, an in-situ fenestration device includes circular cutting staple 50 and a backplate catheter having a backplate as described herein. FIG. 2B depicts a perspective view of circular cutting staple 50 in a deformed position within fenestration 52 in stent material 54. While cutting staple 50 shown in FIGS. 2A and 2B is circular, the cutting staple may have other curved shapes. As shown herein, circular cutting staple 50 may be delivered in a steerable catheter to a fenestration site via transfemoral access, however, access may also be provided via radial or carotid access. Circular cutting staple 50 may be advanced mechanically or hydraulically. The backplate may be delivered in the backplate catheter to the fenestration site via a side branch artery (e.g., the left subclavian artery) of an aortic arch. If the steerable catheter is delivered via radial or carotid access, the backplate may be delivered transfemorally. Circular cutting staple 50 includes cutting edge 56 at the distal end thereof. Cutting edge 56 is configured to cut graft material to create a fenestration. After the cutting operation is performed, circular cutting staple 50 is configured to deform from the cutting position to the deformed position to capture frayed edges of the fenestration. Circular cutting staple 50 may be formed of a metal material such as a shape memory metal material (e.g., Nitinol).

As shown in FIG. 2A, cutting edge 56 extends 360 degrees around the distal end of circular cutting staple 50. In other embodiments, the cutting edge may extend less than 360 degrees (e.g., the cutting edge may have one or more discontinuities along the distal end of the circular cutting staple). The catheter delivering the backplate of the circular cutting staple may be configured to capture and remove the cut material from the fenestration site. The fenestration cut by circular cutting staple 50 may be sized to permit blood flow from an aortic arch to a side branch artery. Circular cutting staple 50, in its deformed position, may be configured to provide a rigid landing zone for a post fenestration branch stent graft. Circular cutting staple 50 may be formed of a material visible on fluoro to provide a visible landing zone for a clinician. The positioning of circular cutting staple 50 may be supported using an electro-magnet or other positioning device. A steerable catheter may be used to support the positioning of circular cutting staple 50. Low-profile catheters (e.g., 12, 13, 14, or 15 French) may be used to deliver circular cutting staple 50 and the backplate.

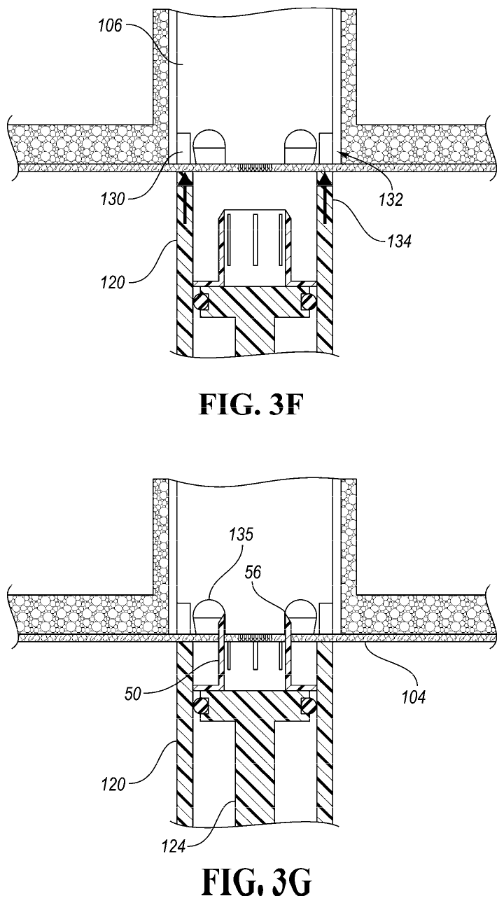

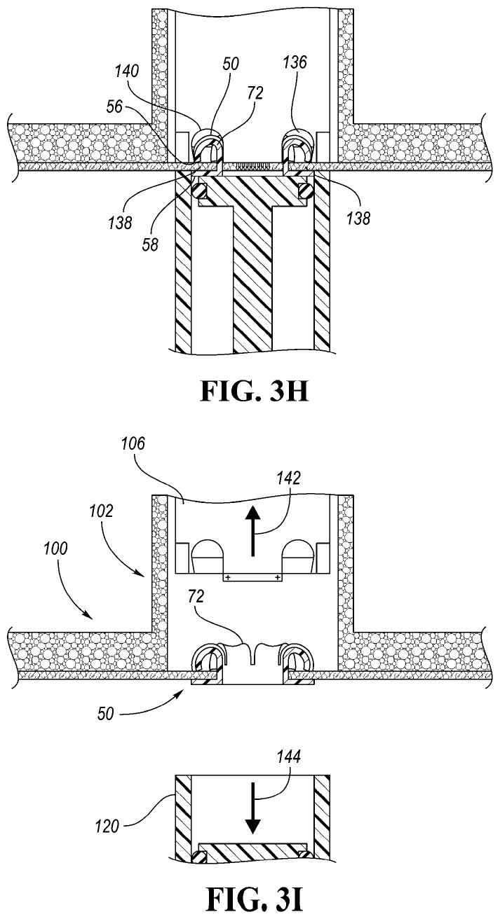

FIG. 3A depicts a partial cut away, schematic, side view of aortic arch 100 and branch artery 102 (e.g., the left subclavian artery) thereof. Stent graft 104 is in a deployed position within aortic arch 100. As shown in FIG. 3A, a portion of stent graft 104 occludes blood flow from aortic arch 100 to branch artery 102. The portion of stent graft 104 may be formed of graft material without any graft stents. An in-situ fenestration system including a circular cutting staple of one or more embodiments is configured to cut the graft material to form a fenestration to provide blood flow from aortic arch 100 to branch artery 102.

FIG. 3B depicts a partial cut away, schematic, side view of backplate catheter 106 including backplate 108 carried at a distal end thereof as part of an in-situ fenestration system according to one or more embodiments. Backplate catheter 106 may be advanced through branch artery 102 (e.g., the left subclavian artery). Backplate catheter 106 may have a diameter of 20, 21, 22, 23, or 24 French. Advancement of backplate catheter 106 may be aided by vessel dilation. FIG. 3C depicts magnified area 3C of FIG. 3B. As shown in FIGS. 3B and 3C, microneedles 110 are carried on backplate 108. In one or more embodiments, microneedles 110 are only carried on a center portion of backplate 108. Microneedles 110 are configured to capture and remove cut material from a fenestration. Microneedles 110 may extend orthogonally to backplate 108. As shown in FIG. 3C, microneedles 110 include base portions 112 and pointed distal portions 114 extending from base portions 112. Undercuts 116 are formed where base portions 112 and pointed distal portions 114 meet. Pointed distal portions 114 are configured to pierce cut graft material. Undercuts 116 are configured to capture the cut graft material around where it is pierced by pointed distal portions 114.

FIG. 3D depicts a partial cut away, schematic, side view of backplate catheter 106 advanced through branch artery 102 such that backplate 108 presses against the graft material of stent graft 104. Arrow 118 depicts backplate catheter 106 advancing from the position shown in FIG. 3C to the position shown in FIG. 3D. In this position, microneedles 110 may engage the graft material through pointed distal portions 114 piercing the graft material.

FIG. 3E depicts a partial cut away, schematic, side view of steerable catheter 120 having distal end 122 facing stent graft 104 and circular cutting staple 50 advancing through steerable catheter 120. Steerable catheter 120 may have a diameter of 20, 21, 22, 23, or 24 French. In one or more embodiments, the diameter of backplate catheter 106 and steerable catheter 120 may be the same. As shown in FIG. 3E, piston 124 is configured to advance through steerable catheter 120. Circular cutting staple 50 contacts distal end 126 of piston 124, thereby advancing as piston 124 advanced through steerable catheter 120. Circular cutting staple 50 includes proximal base 58 having a surface interfacing with distal end 126 to provide a contacting region between circular cutting staple 50 and piston 124. The circumference of proximal base 58 may be about equal to the inner diameter of steerable catheter 120 to resist radial movement of circular cutting staple 50 relative to steerable catheter 120. Piston 124 includes peripheral channel 125 configured to receive O-ring 128. O-ring 128 is configured to reduce friction as piston 124 advances through steerable catheter 120. As shown in FIG. 3E, circular cutting staple 50 is advanced close to distal end 122 of steerable catheter 120 during this operation.

FIG. 3F depicts a partial cut away, schematic, side view of steerable catheter 120 fixating to backplate catheter 106. In the embodiment shown in FIG. 3F, backplate catheter 106 includes electromagnet 130 positioned within distal end 132 of backplate catheter 106. The electromagnet may extend completely around backplate catheter 106 or may be formed of electromagnetic components placed at discreet positions around distal end 132. Steerable catheter 120 may include a magnetic component (e.g., a magnetic capsule) configured to be attracted to electromagnet 130 as shown by arrows 134 when electromagnet 130 is activated. Steerable catheter 120 may include a capsule made from stainless steel having iron as the magnetic component. Activation of electromagnet 130 aids in positioning of the fenestration site and fixing steerable catheter 120 and backplate catheter 106 for the cutting and stapling operations. In other embodiments, distal ends 122 and 132 of steerable catheter 120 and backplate catheter 106 may be fixated and positioned relative to each other using releasable mechanical features (e.g., barbs, threads, etc.) instead of electromagnet 130 and magnetic components. In one or more embodiments, the electromagnets may be carried within distal end 122 of steerable catheter 120 and the magnetic components may be carried within distal end 132 of backplate catheter 106.

FIG. 3G depicts a partial cut away, schematic, side view of piston 124 advancing through steerable catheter 120 to advance circular cutting staple 50 so that cutting edge 56 cuts (e.g., shears) graft material of stent graft 104 at the fenestration site. Piston 124 may be advanced using hydraulic pressure. Alternatively, piston 124 may be advanced manually. After cutting edge 56 of circular cutting staple 50 cuts through graft material, cutting edge 56 enters channel 136 of backplate catheter 106. As shown in FIG. 2A, cutting edge 56 outwardly tapers from leading edge 60 toward base 62 so that the cut graft material slides along slanted region 64 of cutting edge 56 to resist snagging. In one or more embodiments, cutting edge 56 extends the entire circumference of circular cutting staple 50, thereby creating a circular cut into the graft material at the fenestration site. A linear cutting motion may be supplemented by rotation of circular cutting staple 50.

FIG. 3H depicts a partial cut away, schematic, side view of circular cutting staple 50 in the deformed state, thereby forming pinch point 138 between cutting edge 56 and proximal base 58 of circular cutting staple 50. As shown in FIG. 2A, circular cutting staple 50 includes medial portion 66 including longitudinal perforations 68 (e.g., slots) extending between leading edge 60 and proximal base 58. Longitudinal perforations 68 may be formed by a laser cutting operation. Longitudinal perforations 68 terminate short of leading edge 60 to form areas of weakness 70. Areas of weakness 70 are configured to tear as leading edge 60 advances against curved surface 140 of channel 136. As areas of weakness 70 tear, fingers 72 are formed between longitudinal perforations 68. Each of fingers 72 have a raised edge 74 and an indented middle portion 76. As shown in FIG. 2A, there are 6 longitudinal perforations and areas of weakness forming 6 fingers. In other embodiments, a different number of pairs of longitudinal perforations and areas of weakness may be used to form a different number of fingers (e.g., 3, 4, 5, 7, 8, 9, 10, 11, or 12). Circular cutting staple 50 is advanced until fingers 72 and proximal base 58 of circular cutting staple 50 forms pinch point 138 to anchor circular cutting staple 50 to the graft material around the periphery of the fenestration hole. In this anchored position, proximal base 58 and each of the fingers 72 contact (e.g., clamp) the graft material. In the anchored position, circular cutting staple 50 is configured to capture frayed edges from the fenestration (thereby mitigating against fraying of the graft material) and/or to provide a rigid landing zone for a branch stent graft. Fingers 72 may be biased to remain in the deployed position.

FIG. 3I depicts a partial cut away, schematic, side view of backplate catheter 106 and steerable catheter 120 retracting through branch artery 102 and aortic arch 100, respectively, and away from deployed circular cutting staple 50 having deformed fingers 72. The retraction of backplate catheter 106 and steerable catheter 120 are represented by arrows 142 and 144, respectively. Before backplate catheter 106 and steerable catheter 120 are retracted, electromagnet 130 is deactivated or the mechanical features are disengaged so that backplate catheter 106 and steerable catheter 120 are not fixated to each other. Cut material 146 from the fenestration is captured on microneedles 110 and is removed along with backplate catheter 106. In one or more embodiments, circular cutting staple 50 remains with the fenestration after backplate catheter 106 and steerable catheter 120 are retracted.

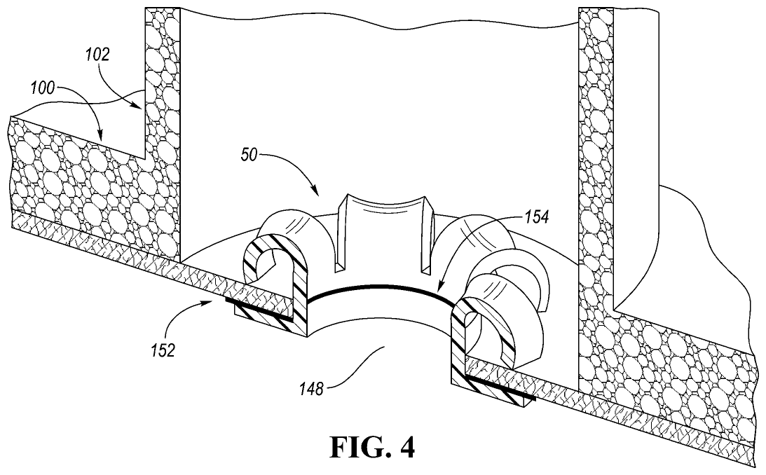

FIG. 4 depicts a schematic, perspective view of circular cutting staple 50 in a deployed position around fenestration 148. As depicted by arrow 150, blood flows from aortic arch 100 to branch artery 102 through fenestration 148. Proximal base 58 of circular cutting staple 50 seals against the graft material in aortic arch 100 as depicted by seal zones 152. The graft material deployed within deployed circular cutting staple 50 seals in branch artery 102 as depicted by seal zone 154. Seal zones 152 and 154 are configured to resist leakage of blood flow outside of the deployed cutting staple 50 and the graft material.

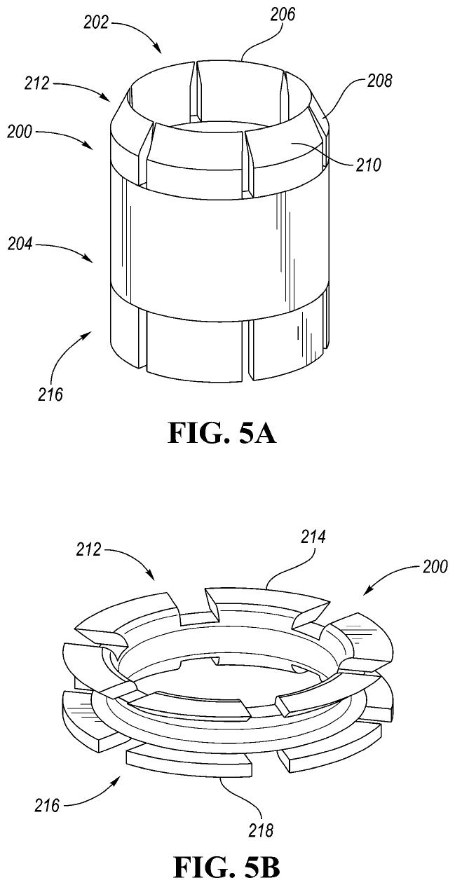

FIG. 5A depicts an isolated, perspective view of circular cutting stent 200 in a cutting position. In one or more embodiments, an in-situ fenestration device includes circular cutting stent 200 and a backplate catheter having a backplate as described herein. FIG. 5B depicts an isolated, perspective view of circular cutting stent 200 in a deployed position. Circular cutting stent 200 may be formed of a shape memory material such as Nitinol. While cutting stent 200 shown in FIGS. 5A and 5B is circular, the cutting stent may have other curved shapes. As shown herein, circular cutting stent 200 may be delivered in a steerable catheter to a fenestration site via transfemoral access. Circular cutting stent 200 includes cutting edge 202 at the distal end thereof. Cutting edge 202 is configured to cut graft material to create a fenestration. After the cutting operation is performed, circular cutting stent changes from the cutting position to the deployed position to capture frayed edges of a fenestration. Distal end 212 of circular cutting stent 200 may have a preformed shape that is restricted in the cutting position. Distal end 212 may take on the preformed shape after the cutting operation as shown in FIG. 5B. Proximal end 216 of circular cutting stent 200 may have a preformed shape that is restricted while circular cutting stent 200 is being delivered. Proximal end 216 may take on the preformed shape when proximal end 216 is no longer restricted as shown in FIG. 5B. Circular cutting stent 200 may include self-expanding region 204. Self-expanding region 204 may be formed of a self-expanding material such as Nitinol. An expanding operation (e.g., with a balloon) may be used to expand the diameter of self-expanding region 204.

As shown in FIG. 5A, cutting edge 202 extends 360 degrees around the distal end of circular cutting stent 200. In other embodiments, the cutting edge may extend less than 360 degrees (e.g., the cutting edge may have one or more discontinuities along the distal end of the circular cutting stent). Circular cutting stent 200, in its deployed position, may be configured to provide a rigid landing zone for a post fenestration branch stent graft. Circular cutting stent 200 may be formed of a material visible on fluoro to provide a visible landing zone for a clinician. A steerable catheter may be used to support the positioning of circular cutting stent 200. Circular cutting stent 200 may be configured for use with a low-profile backplate catheter (e.g., 12, 13, 14, or 15 French).

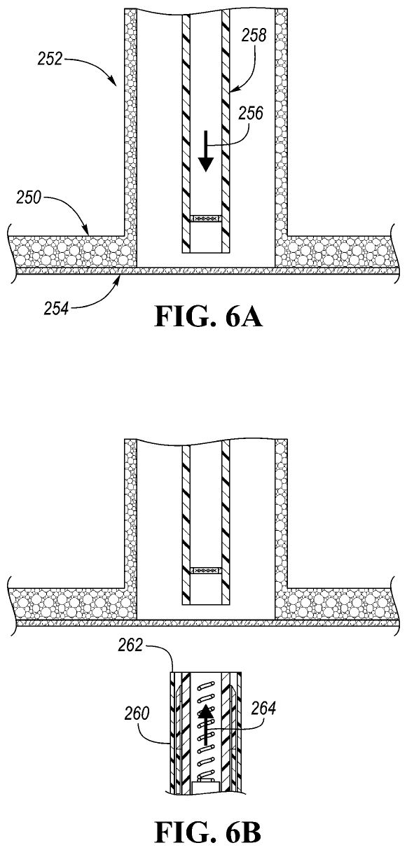

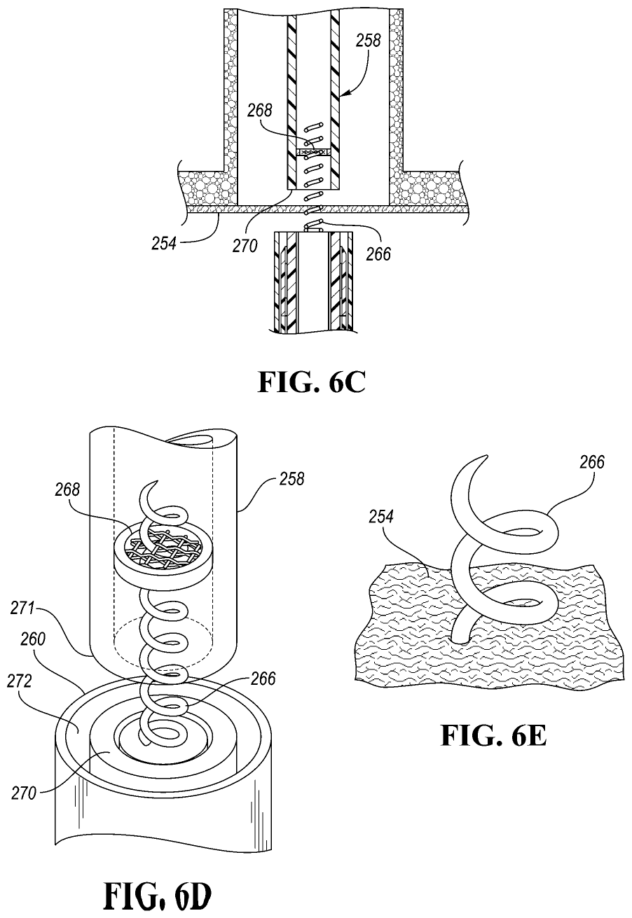

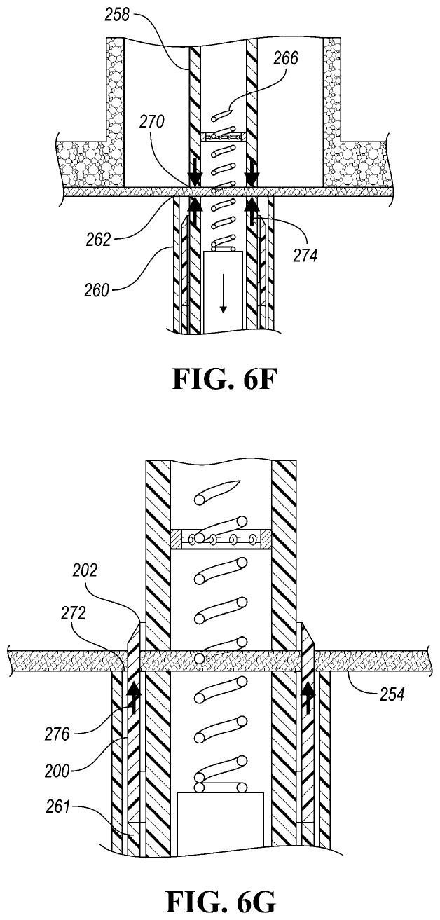

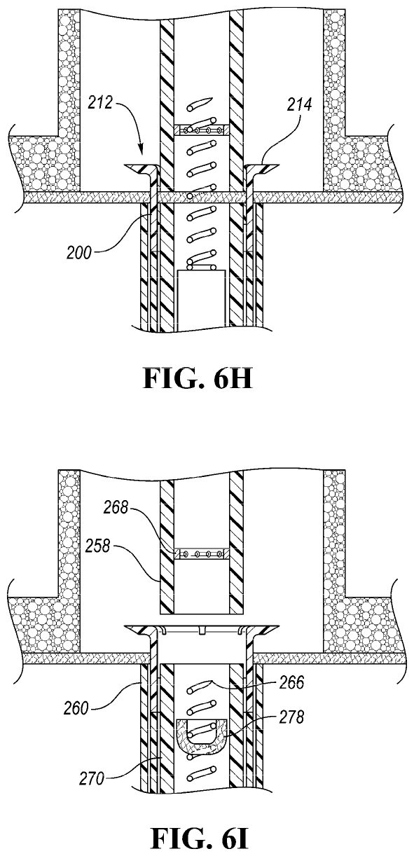

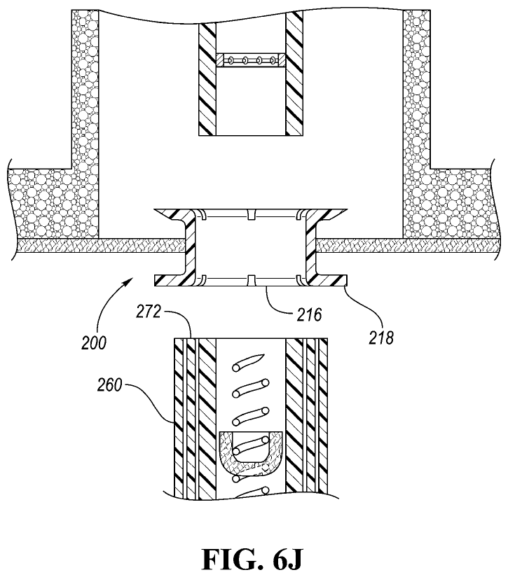

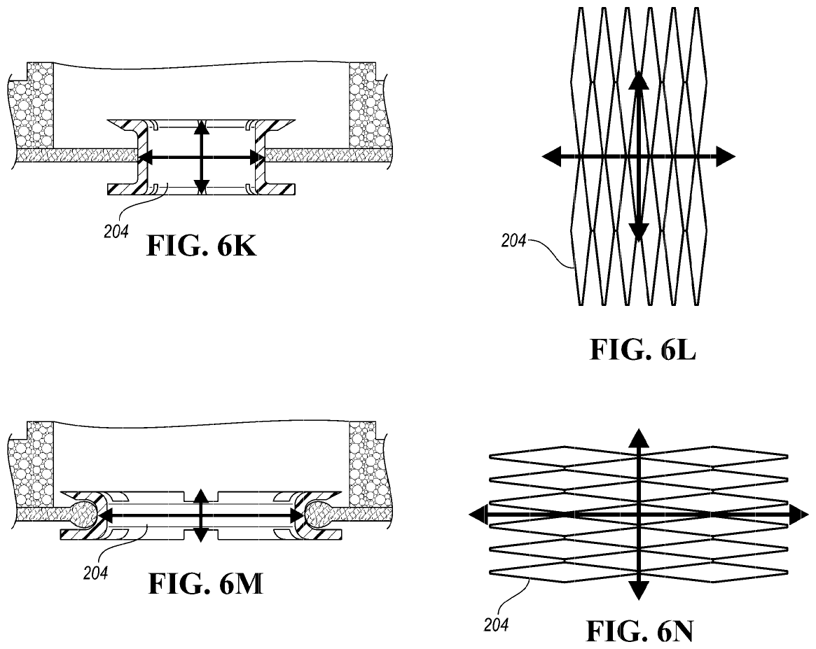

FIG. 6A depicts a partial cut away, schematic, side view of aortic arch 250 and branch artery 252 (e.g., the left subclavian artery) extending therefrom. Stent graft 254 is in a deployed position within aortic arch 250. As shown in FIG. 6A, a portion of stent graft 254 occludes blood flow from aortic arch 250 to branch artery 252. The portion of stent graft 254 may be formed of graft material without any graft stents. However, in other embodiments, backplate catheter 258 may be advanced transfemorally and the steerable catheter 260 may be advanced through branch artery 252. As represented by arrow 256, backplate catheter 258 advances through branch artery 252 (e.g., the left subclavian artery). However, in other embodiments, backplate catheter 258 may be advanced transfemorally and the steerable catheter 260 may be advanced through branch artery 252. Backplate catheter 258 may be a low-profile backplate catheter (e.g., 12, 13, 14, or 15 French).

FIG. 6B depicts a partial cut away, schematic, side view of steerable catheter 260 having distal end 262 facing stent graft 254. Steerable catheter 260 is advanced proximal to branch artery 252 using a steering operation guided by fluoro. Arrow 264 depicts the advancement of steerable catheter 260. Backplate catheter 258 may have any of the following diameters: 8, 9, 10, 11, or 12 French. Steerable catheter 260 may have any of the following diameters: 15, 16, 17, 18, 19, or 20 French.

FIG. 6C depicts a partial cut away, schematic, side view of rotatable coil 266 advancing via rotation through the stent graft material of stent graft 254 and backplate 268 of backplate catheter 258. FIG. 6D depicts a fragmented, schematic view of steerable catheter 260, backplate catheter 258, and rotatable coil 266 extending into backplate catheter 258 from steerable catheter 260. Inner catheter 270 extends within steerable catheter 260. Rotatable coil 266 is configured to track through inner lumen of inner catheter 270. Outer lumen 272 is formed between steerable catheter 260 and inner catheter 270. FIG. 6E depicts a fragmented, schematic view of graft material of stent graft 254 with rotatable coil 266 rotating therethrough. As shown in FIG. 6C, backplate 268 is proximally offset from distal end 271 of backplate catheter 258. In one or more embodiments, backplate 268 is formed of a rigid mesh material (e.g., a rigid metal mesh material) having gaps suitable for rotatable coil 266 to pass through during rotation thereof. Rotatable coil 266 is also configured to rotate through stent graft material (e.g., fabric material) through rotation thereof.

FIG. 6F depicts a partial cut away, schematic, side view of rotatable coil 266 being pulled linearly in a proximal direction as depicted by arrow 273 to pull the distal ends 262 and 270 of steerable catheter 260 and backplate catheter 258, respectively, together and fix the position of steerable catheter 260 and backplate 268 relative to each other. When pulled together, distal ends 262 and 270 of steerable catheter 260 and backplate catheter 258, respectively, pinch the graft material to be cut to from a fenestration as depicted by arrows 274.

FIG. 6G depicts a partial cut away, schematic, side view of circular cutting stent 200 in a cutting position advancing as represented by arrows 276 from outer lumen 272 so that cutting edge 202 cuts (e.g., shears) graft material of stent graft 254 at the fenestration site. Circular cutting stent 200 may be advanced relative to steerable catheter 260 by advancing sheath 261 located within outer lumen 272. As shown in FIG. 5A, cutting edge 202 outwardly tapers from leading edge 206 toward base 208 so that the cut graft material slides along slanted region 210 of cutting edge 202 to resist snagging. In one or more embodiments, cutting edge 202 extends the entire circumference of circular cutting stent 200, thereby creating a circular cut into the graft material at the fenestration site. A linear cutting motion may be supplemented by rotation of circular cutting stent 200.

FIG. 6H depicts a partial cut away, schematic, side view of circular cutting stent 200 in a partially deployed position where distal end 212 has taken on a preformed shape. The preformed shape includes distal tabs 214. As shown in FIG. 5B, circular cutting stent 200 includes 6 distal tabs 214. In other embodiments, a different number of distal tabs may be used (e.g., 3, 4, 5, 7, 8, 9, 10, 11, or 12).

FIG. 6I depicts a partial cut away, schematic, side view of rotatable coil 266 rotating in a proximal direction to disengage rotatable coil 266 from backplate 268, thereby disconnecting steerable catheter 260 from backplate catheter 258. Once rotatable coil 266 is disengaged from backplate 268, rotatable coil may be rotated in a proximal direction or translated in a linear proximal direction to pull cut material 278 within inner catheter 270, as shown in FIG. 6I.

FIG. 6J depicts a partial cut away, schematic, side view of circular cutting stent 200 in a deployed position where proximal end 216 has taken on a preformed shape. The preformed shape includes proximal tabs 218. As shown in FIG. 5B, circular cutting stent 200 includes 6 proximal tabs 218. In other embodiments, a different number of proximal tabs may be used (e.g., 3, 4, 5, 7, 8, 9, 10, 11, or 12). Proximal end 216 takes on the preformed shape with proximal tabs 218 when steerable catheter 260 is retracted relative to circular cutting stent 200 so that proximal end 216 is no longer retracted within outer lumen 272.

Self-expanding region 204 of circular cutting stent 200 may expand using a pre-set shape of a shape memory material (the pre-set shape is achieved when self-expanding region 204 is no longer restricted) or through balloon expansion of a mesh material (after circular cutting stent 200 is deployed within a fenestration). FIGS. 6K and 6L depict a schematic, side view and a schematic, isolated view, respectively, of self-expanding region 204 before it has expanded. FIGS. 6M and 6N depict a schematic, side view and a schematic, isolated view, respectively, of self-expanding region 204 after expansion. By expanding in the radial direction, the axial direction is contracted. In one or more embodiments, this simultaneously expands the fenestration and allows the tabs to clamp the graft material.

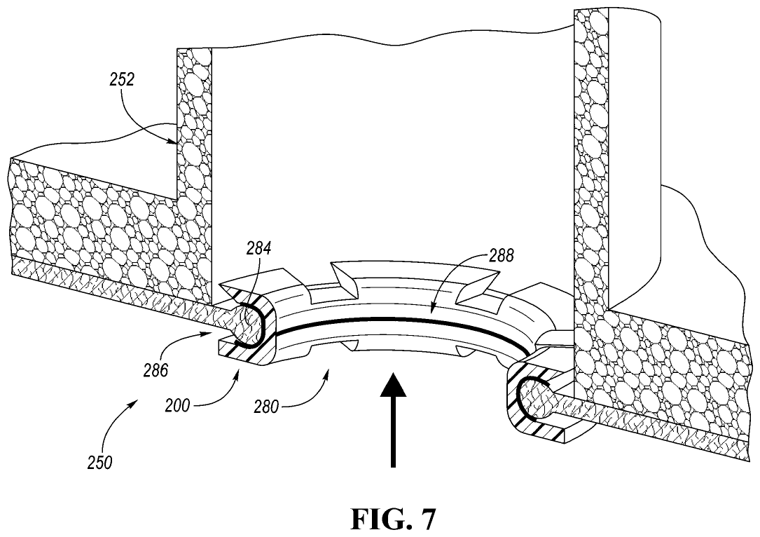

FIG. 7 depicts a schematic, perspective view of circular cutting stent 200 in a deployed position around fenestration 280. As depicted by arrow 282, blood flows from aortic arch 250 to branch artery 252 through fenestration 280. Pinch point 284 seals against the graft material in aortic arch 250 as depicted by seal zone 286. The graft material deployed within deployed circular cutting stent 200 seals in branch artery 252 as depicted by seal zone 288. Seal zones 286 and 288 are configured to resist leakage of blood flow outside of the deployed cutting stent 200 and the graft material.

In one or more embodiments, the backplate may be delivered through the transfemoral pathway or the subclavian pathway and the cutting staple or stent may be delivered through the other of the transfemoral pathway and the subclavian pathway. The cutting staple may be hydraulically actuated. The cutting stent may be mechanically actuated. The cutting staple or stent may be housed within a capsule of a steerable catheter until the device is ready for deployment. The fenestration formed by the cutting staple or stent may be any of the following values or in a range of any two of the following values: 7.5, 7.6, 7.7, 7.8, 7.9, 8.0, 8.1, 8.2, 8.3, 8.4, or 8.5 millimeters. The cutting staple may be formed of a cobalt chromium material. In one or more embodiments, the force to yield one staple finger may be 12, 13, or 14 Newton. In one or more embodiments, the force to yield six staple fingers may be 75, 80, or 85 Newtons.

In one embodiment, an in-situ fenestration device for forming a fenestration in graft material of a deployed stent graft is disclosed. The in-situ fenestration device may include an expanding fenestration needle including a plurality of discrete needles forming an outer surface of the expanding fenestration needle. The in-situ fenestration device may further include a balloon in communication with the plurality of discrete needles. The balloon is configured to expand to transition the discrete needles from a delivery position to a deployed position to form the fenestration in the graft material of the deployed stent graft.

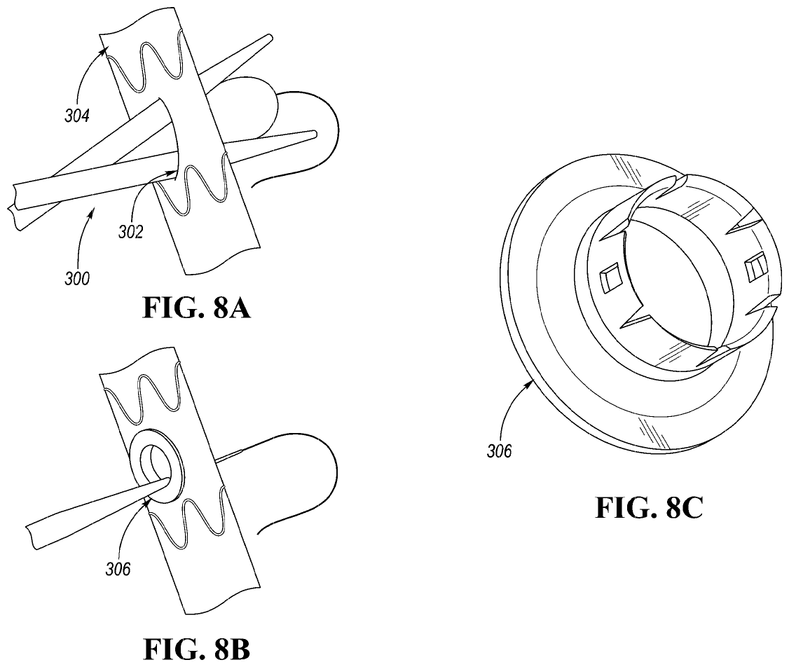

FIG. 8A depicts a perspective, schematic view of fenestration needle 300 configured to create fenestration 302 in deployed stent graft 304. In one or more embodiments, fenestration needle 300 is configured to expand using a balloon as described herein. FIG. 8B depicts a perspective, schematic view of grommet 306 deployed within fenestration 302. FIG. 8C depicts a perspective, isolated, schematic view of grommet 306.

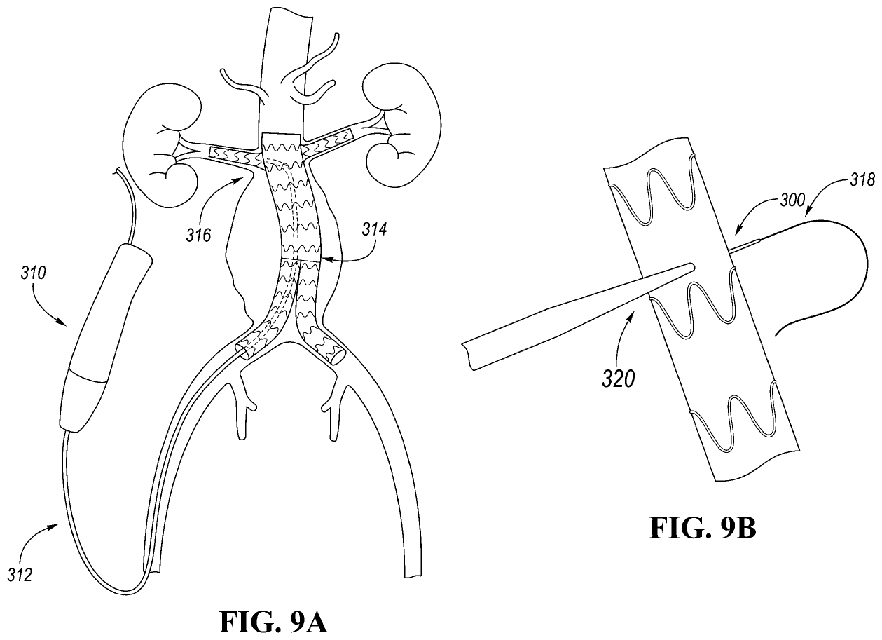

FIG. 9A depicts an isolated, schematic view of in-situ fenestration system 310 including sheath 312 advanced through deployed aortic stent graft 314 to renal artery location 316. FIG. 9B depicts a perspective, schematic view of sheath 312 tracked over guidewire 318 to renal artery location 316. Distal region 320 of sheath 312 includes fenestration needle 300, which may be sharpened or pointed, configured to puncture stent graft at renal artery location 316 to form an initial puncture. Fenestration needle 300 may be vibrated at a high frequency (e.g., ultrasonic) to help it pierce the graft material.

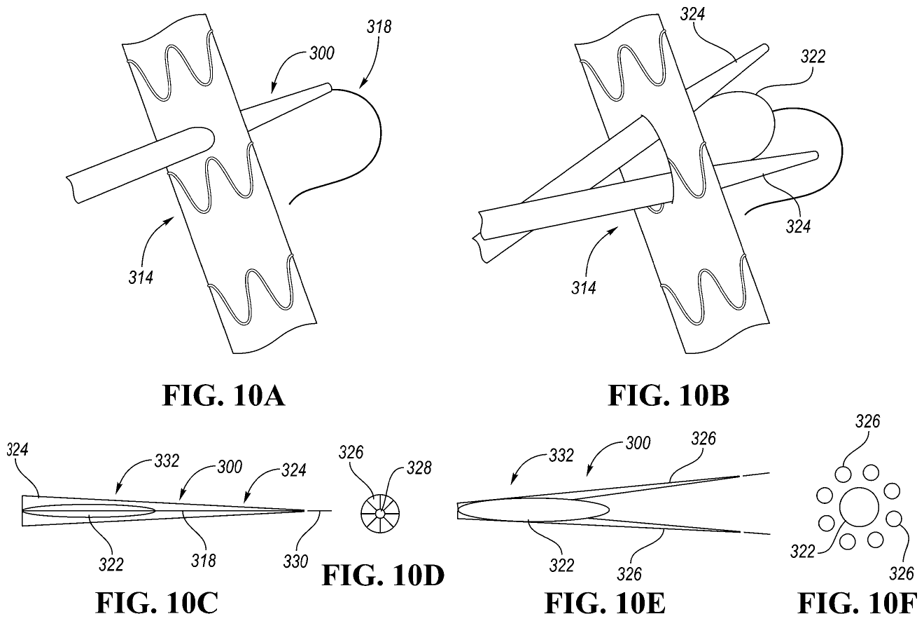

FIG. 10A depicts a perspective, schematic view of fenestration needle 300 advancing through the initial puncture in stent graft 314 to increase the size of the fenestration. FIG. 10B depicts a perspective, schematic view of fenestration needle 300 where balloon 322 radially expands discrete needles 326 of fenestration needle 300 to displace the graft material of stent graft 314 to further increase the size of the fenestration.

FIGS. 10C and 10D depict a cross-section, side view and front view, respectively, of fenestration needle 300 in a delivery position. Fenestration needle 300 includes distal tip 324. In one or more embodiments, distal tip 324 tapers to a narrow end with a circumference sized to penetrate (as opposed to tearing) graft material through the interstitial regions of the sew pattern of the graft material. As shown in FIG. 10D, distal tip 324 is comprised of discrete needles 326 converging at distal tip 324 while forming a channel 328 so that fenestration needle 300 is configured to track over guidewire 318. Each of discrete needles 326 tapers from a narrow end at distal tip 324 to a broader end in a proximal direction. In the embodiment shown in FIG. 10D, there are 8 discrete needles 326. In other embodiments, a different number of discrete needles may be utilized (e.g., 3, 4, 5, 6, 7, 9, 10, 11, or 12). One or more discrete needles 326 may have a smaller profile penetrating needle 330 extending therefrom in a distal direction. The smaller profile of penetrating needle 330 may be sized to fit within graft fibers, while the proximal section 332 of fenestration needle 300 is configured to transmit separating force to discrete needles 326.

FIGS. 10E and 10F depict a cross-section, side view and front view, respectively, of fenestration needle 300 in a deployed position. Once fenestration needle 300 penetrates stent graft 314 as shown in FIG. 10A, balloon 322 residing within proximal section 332 may inflate to separate distal tip 324 into discrete needles 326 extending into a radial configuration shown in FIG. 10F. Balloon 322 may inflate by filling it with a fluid (e.g., gas or liquid such as saline solution). Balloon 322 may be sealed or compartmentalized relative to guidewire 318 so that it can expand upon being filled with liquid. The radial force from balloon 322 transferred to discrete needles 326 pushes the graft material of stent graft 314 radially outward to form the fenestration. In one or more embodiments, the fenestration is formed in this manner without tearing or minimal tearing (e.g., displacing graft material). To the extent tearing occurs, a reinforcing grommet as disclosed herein may be applied to mitigate any negative effects (e.g., leakage and/or further fraying) at the fenestration site.

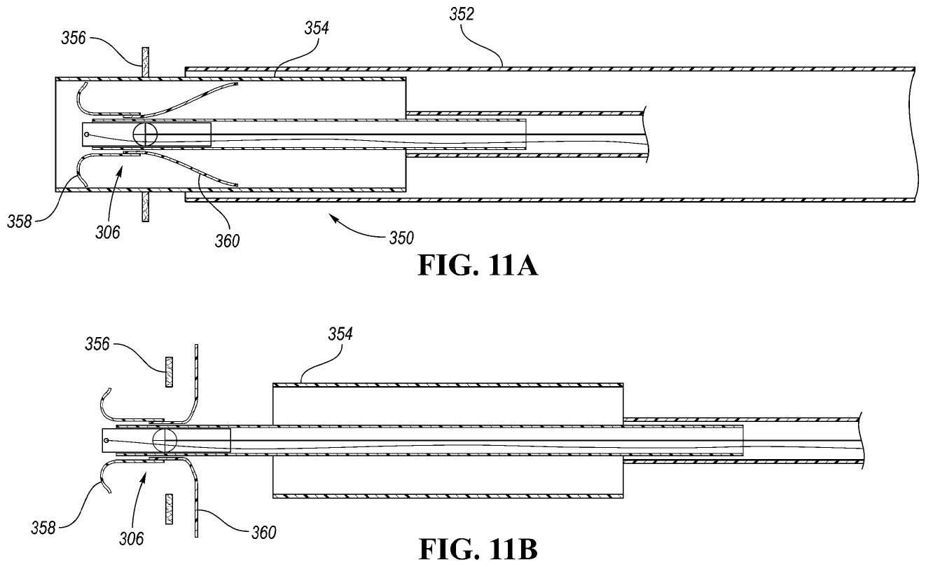

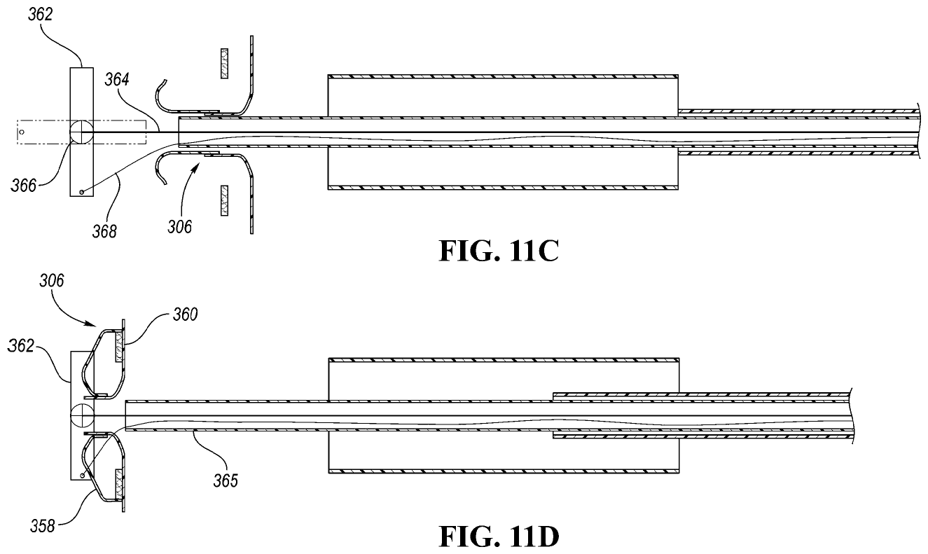

FIGS. 11A through 11D depict cut away schematic, side views of operations of grommet delivery system 350 for reinforcing a fenestration with grommet 306.

As shown in FIG. 11A, grommet delivery system 350 includes sheath 352 and capsule/shaft 354. Sheath 352 tracks over guidewire 318 (as shown in FIG. 10A after fenestration needle 300 has been removed from a patient's vasculature). Capsule/shaft 354 is tracked such that fenestrated graft material 356 straddles first and second sections 358 and 360 of grommet 306. This positioning of first and second sections 358 and 360 of grommet 306 may be aided by placing radiopaque markers on capsule/shaft 354. First section 358 of grommet 306 may be formed of a metal material such as stainless steel. Second section 360 of grommet 306 may be formed of a shape memory material such as Nitinol.

As shown in FIG. 11B, once fenestrated graft material 356 straddles first and second regions 358 and 360 of grommet 306, sheath 352 (as shown in FIG. 11A) is removed and capsule/shaft 354 is retracted to expose second section 360, which extends radially outward to form a base of grommet 306. In its radially extended position, second section 360 may be parallel or generally parallel to fenestrated graft material 356 to support fenestrated graft material 356.

As shown in FIG. 11C, rotatable force member 362, in an unrotated position, is advanced from and out of the lumen formed by grommet 306 via force wire 364. Once rotatable force member 362 clears the lumen formed by grommet 306, rotatable force member 362 is rotated about pivot axis 366 via rotation wire 368 into a rotated position. In one or more embodiments, pivot axis 366 is perpendicular to longitudinal axis of capsule/shaft 354. In one or more embodiments, the angle between the unrotated and rotated positions is 90 degrees.

As shown in FIG. 11D, rotatable force member 362, in the rotated position, is retracted with a relatively high force to deform first section 358 of grommet 306 and to clamp fenestrated graft material 356 between first and second sections 358 and 360 of grommet 306, thereby supporting the fenestrated graft material 356. Once grommet 306 clamps fenestrated graft material 356, hypotube 365 is released from grommet 306 and retracted. In one or more embodiments, spring actuated grippers connect grommet 306 and hypotube 365. The spring actuated grippers are released before retracting hypotube 365.

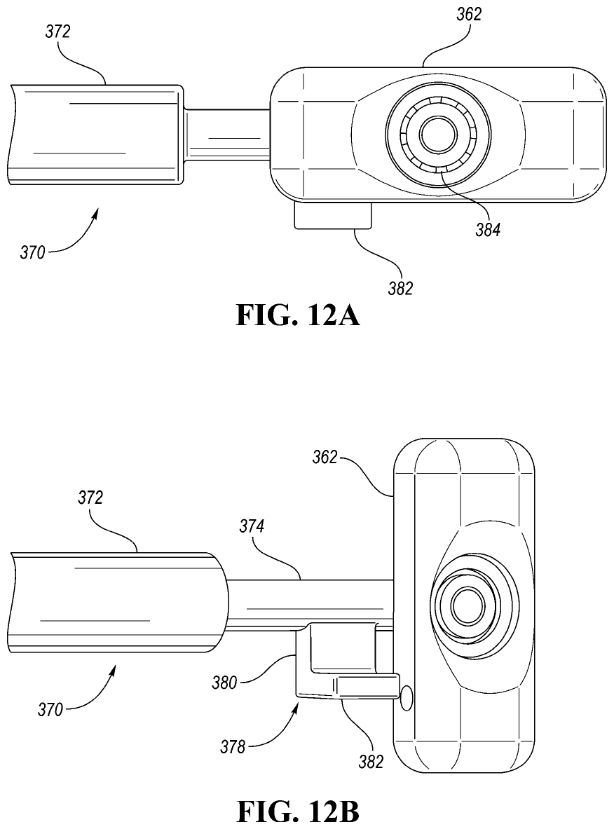

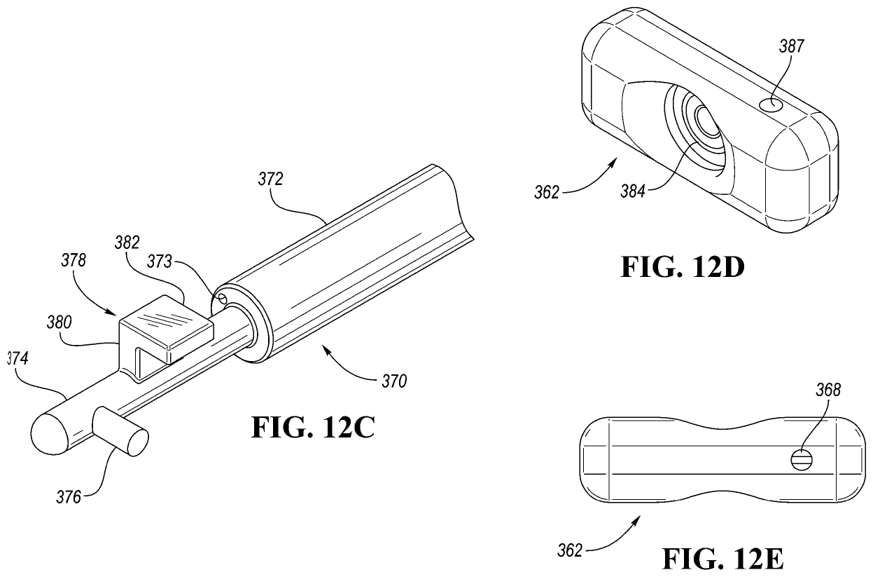

FIG. 12A depicts a schematic, plan view of rotating member system 370 including rotatable force member 362. FIG. 12A depicts rotatable force member 362 in an unrotated position. FIG. 12B depicts a schematic, plan, perspective view of rotating member system 370 including rotatable force member 362. FIG. 12B depicts rotatable force member 362 in a rotated position. FIG. 12C depicts a schematic, perspective view of rotating member system 370 with rotatable force member 362 removed.

Rotatable member system 370 includes compression shaft 372 having lumen 373 for force wire 364 (e.g., pull wire). Compression shaft 372 may be formed of an extruded material (e.g., a plastic or metal extruded material). Rotation shaft 374 extends from the distal end of compression shaft 372. Rotation shaft 374 is configured to rotate about its longitudinal axis. Rotation shaft 374 may be machined from a metal material. Rotation shaft 374 includes bearing shaft 376 and stop 378. Bearing shaft 376 extends orthogonally from rotation shaft 374. Stop 378 includes base portion 380 extending from rotation shaft 374 and extending portion 382 extending from base portion 380. In one or more embodiments, base portion 380 and extending portion 382 may be orthogonal. Stop 378 is disposed between distal end of compression shaft 372 and bearing shaft 376. In the unrotated position, rotatable force member 362 stops against a surface of extending portion 382 of stop 378 as shown in FIG. 12A. In the rotated position, rotatable force member 362 stops against a side surface of extending portion 382 of stop 378 as shown in FIG. 12B.

FIG. 12D depicts a perspective, isolated view of rotatable force member 362. FIG. 12E depicts a top, isolated view of rotatable force member 362 including rotation wire 368 within channel 387. Rotatable force member 362 may be machined from an electro-polished stainless steel or other suitable metal material. Rotatable force member 362 includes ball bearing 384, which bears against bearing shaft 376 as rotatable force member 362 rotates from the unrotated position to the rotated position. Ball bearing 384 may be shielded for biocompatibility.

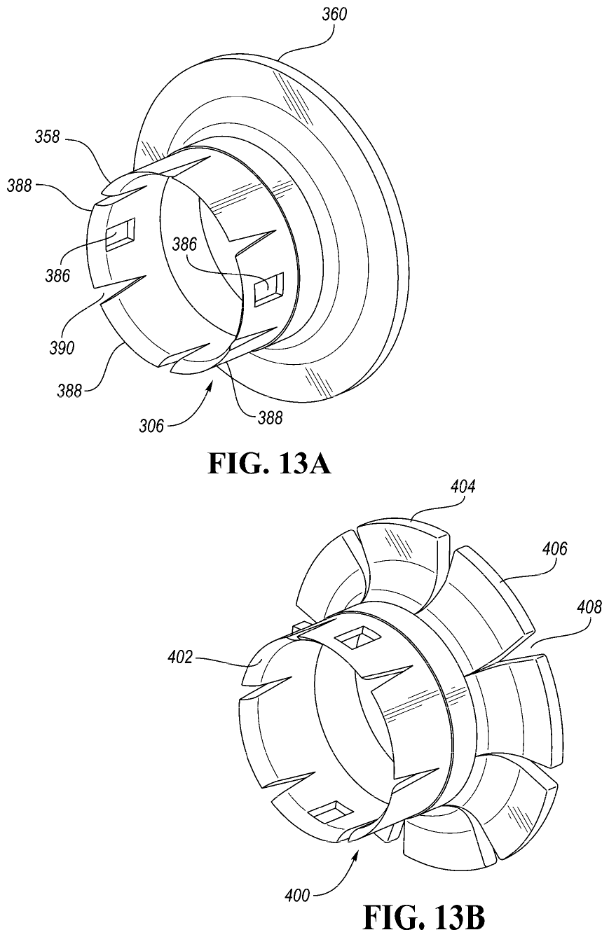

FIG. 13A depicts a perspective, schematic view of grommet 306 including first section 358 and second section 360. Second section 360 is shown in a radially expanded position after it is separated from capsule/shaft 354. First section 358 and second section 360 may be formed of first and second metal materials, respectively, welded together with a third metal material. The third metal material may be an intermediate metal material that allows for otherwise incompatible, different first and second metal materials. For example, niobium may be used to weld a first metal material of stainless steel and a second metal material of Nitinol. First and/or second sections 358 and 360 may coated with a radiopaque coating to aid in locating branch stents within grommet 306.

First section 358 of grommet 306 may include hook windows 386 configured to attach hooks allowing for controlled release of grommet 306 from grommet delivery system 350. In one or more embodiments, the hooks may be designed to break in grommet 306 instead of breaking off hook windows 386. Although 2 hook windows are shown in FIG. 13A, in other embodiments, first section 358 of grommet 306 may include 1, 3, 4, 5, or 6 hook windows.

First section 358 of grommet 306 may include flanged portions 388 separated by slits 390. First section 358 may be formed of a plastically deformable, biocompatible material (e.g., stainless steel) that is suitable for forming flanged portions 388 with rotating member system 370 including rotatable force member 362. Flange portions 388 may be bent by rotatable force member 362, which may be actuated through a lumen of grommet delivery system. Flange design may be altered to accommodate force member design. For example, while 6 flanged portions are shown in FIG. 13A, in other embodiments, due to space consideration, there may be 1, 2, 3, 4, 5, 7, or 8 flanges.

FIG. 13B depicts a perspective, schematic view of grommet 400 including first and second sections 402 and 404 where second section 404 includes flanges 406 separated by slits 408. Second section 404 may be cut from a metal material sheet (e.g., a Nitinol sheet) at a desired thickness to provide structural integrity when first section 402 is deformed to crimp around fenestrated graft material. First section 402 may be formed of stainless steel. Slits 408 may be utilized to balance structural reinforcement with crimping force. Second section 404 may include any of the following number of flange/slit pairs: 1, 2, 3, 4, 5, 6, 7, 8, 9, 10, 11, and 12.

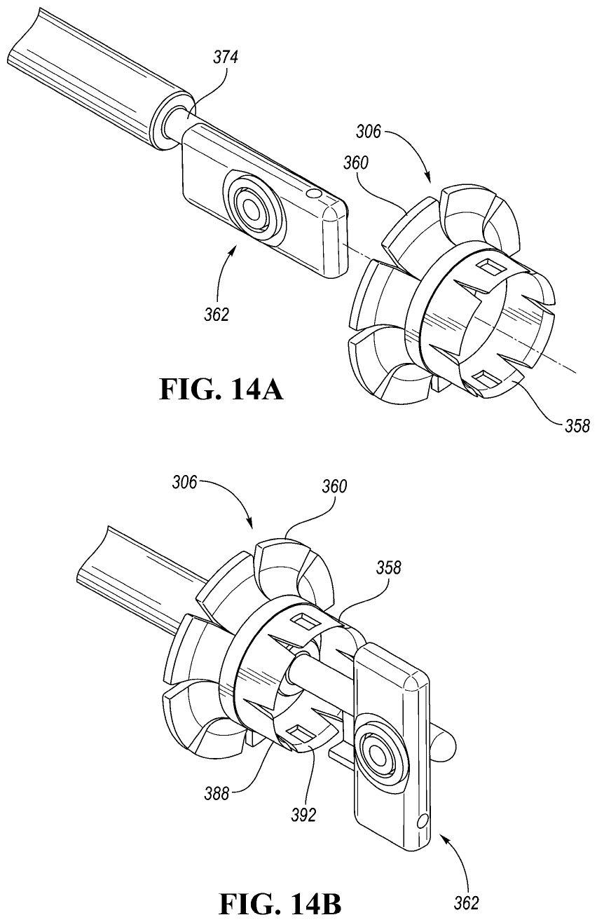

FIG. 14A depicts a perspective, schematic view of rotatable force member 362 proximal to the lumen of grommet 306 and in the unrotated position. FIG. 14B depicts a perspective, schematic view of rotatable force member 362 after it advances through the lumen of grommet 306 and transitions from the unrotated position to the rotated position. Once in the rotated position, rotatable force member 362 may be retracted to contact flange portions 388 to crimp them outward to clamp fenestrated graft material between the outwardly crimped flange portions 388 and second section 360. The rotatable force member 362 is then advanced to clear the rest of first section 358. The axial orientation of rotatable force member 362 may be changed by rotating rotation shaft 374 to align the rotatable force member 362 with uncrimped flange portions 388 so that those flange portions may be crimped. These operations may be repeated until all (or a portion thereof) of the flange portions 388 are crimped. At this point in the process, the rotatable force member 362 may be advanced and then rotated to the unrotated position so that it can be removed through the lumen of grommet 306. Flange portions 388 include inwardly tapered ramps 392 configured to interface with rotatable force member 362 to provide a ramped surface to aid in the crimping operation. One or more of the flanges of second section 360 may align with one or more crimped flange portions 388 of first section 358 to reinforce the structural integrity of the crimp around the fenestrated graft material.

The expanding fenestration needles and/or crimping grommets of one or more embodiments may have one or more of the following benefits. The expanding fenestration needles and/or crimping grommets may fit a wide range of anatomies. The crimping grommets of one or more embodiments may accommodate a fenestration technique that does not result in a clean edge (e.g., the crimping grommets may address frayed edges). In one or more embodiments, the grommet may be applied once blood flow has been established. A branch stent may be relatively easy to place with imaging markers on the grommet. Balloon expansion for the expanding fenestration needle may allow for radial expansion proportional to the amount of air pressure applied to the balloon.

The detailed description set forth herein includes several embodiments where each of the embodiments include several components, features, and/or steps. For the avoidance of doubt, any component, feature, and/or step of one embodiment may be applied, mixed, substituted, matched, and/or combined with one or more components, features, and/or steps of other embodiments. Such resulting embodiments are expressly within the scope of this disclosure. For example, any systems and methods for locating a branch ostium of a branch vessel disclosed herein may be used in conjunction with any disclosed embodiments. Similarly, any systems, methods, or energy types for creating a fenestration (e.g., heat, laser, vibration, RF energy, blades/mechanical cutting) may be used in any disclosed embodiments. In any of the embodiments disclosed herein, following the creation of a fenestration the fenestration may be reinforced or strengthened by placing a stent or grommet like device in the fenestration. After a fenestration is created (and optionally reinforced), a branch stent graft may be tracked and deployed within the fenestration using a separate delivery system. The branch stent graft may extend within the fenestration and at least partially within a main lumen of the fenestrated stent graft and into branch artery (e.g., renal artery, celiac, SMA, BCA, LCC, LSA, etc.). The systems, methods, and devices disclosed herein may be used to make multiple fenestrations in a single stent graft, which thereafter each receive a branch stent graft. Vacuum aspiration may be used in conjunction with any of the devices or methods described herein to remove potential emboli (e.g., gas or pieces of graft material) from the fenestration site. Vacuum aspiration may be applied through a lumen of the fenestration system or may be provided by a standalone aspiration catheter.

While exemplary embodiments are described above, it is not intended that these embodiments describe all possible forms. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. An in-situ fenestration device comprising:
a cutting staple having a delivery position, a cutting position, and a deployed position;
a delivery catheter configured to deliver the cutting staple to an in-situ fenestration site of a graft material situated within an artery; and
a backplate catheter fixating to the delivery catheter as the cutting staple transitions from the delivery position to the cutting position to cut an in-situ fenestration at the in-situ fenestration site, the backplate catheter includes one or more channels configured to transition the cutting staple from the cutting position to the deployed position anchoring the cutting staple to the graft material around a periphery of the in-situ fenestration.

2. The in-situ fenestration device of claim 1, wherein the backplate catheter includes a backplate carrying microneedles configured to capture and to remove the graft material cut by cutting staple in the cutting position.

3. The in-situ fenestration device of claim 2, wherein the microneedles include base portions and pointed distal portions extending from the base portions, the microneedles further include undercuts formed where the base portions and the pointed distal portions meet, the pointed distal portions are configured to pierce the graft material, and the undercuts are configured to capture the cut graft material around where it is pierced by the pointed distal portions.

4. The in-situ fenestration device of claim 1 further comprising a piston configured to advance the cutting staple within the delivery catheter, wherein the piston includes a peripheral channel configured to receive an O-ring configured to reduce friction as the piston advances the cutting staple through the delivery catheter.

5. The in-situ fenestration device of claim 4, wherein the cutting staple includes a proximal base engaging the piston as the piston advances the cutting staple through the delivery catheter.

6. The in-situ fenestration device of claim 1, wherein the backplate catheter includes an electromagnet and the delivery catheter includes a magnetic component configured to be attracted to the electromagnet.

7. The in-situ fenestration device of claim 6, wherein the electromagnet is carried within a distal end of the backplate catheter and the magnetic component is carried within a distal end of the delivery catheter.

8. The in-situ fenestration device of claim 1, wherein the one or more channels include one or more curved surfaces configured to transition the cutting staple from the cutting position to the deployed position.

9. An in-situ fenestration cutting staple comprising:
a proximal base;
a leading edge;
a medial portion extending from the proximal base toward the leading edge; and
a cutting edge outwardly tapering from the leading edge toward the medial portion, the cutting edge configured to cut into a graft material at an in-situ fenestration site to form an in-situ fenestration.

10. The in-situ fenestration cutting staple of claim 9, wherein the medial portion includes longitudinal perforations extending between the leading edge and the proximal base to form areas of weakness configured to tear when an opposing force is applied to the leading edge to form fingers configured to pinch the graft material at a periphery of the in-situ fenestration in an anchored position.

11. The in-situ fenestration cutting staple of claim 10, wherein the proximal base and the fingers contact the graft material in the anchored position.

12. The in-situ fenestration cutting staple of claim 10, wherein each of the fingers have a raised edge and an indented portion in the anchored position.

13. The in-situ fenestration cutting staple of claim 10, wherein adjacent pairs of the fingers are spaced apart from each other.

14. The in-situ fenestration cutting staple of claim 10, wherein the longitudinal perforations terminate short of the leading edge.

15. The in-situ fenestration cutting staple of claim 10, wherein the longitudinal perforations terminate short of the proximal base.

16. The in-situ fenestration cutting staple of claim 9, wherein the cutting staple is a circular cutting staple, the cutting edge extends an entire circumference of the circular cutting staple to create a circular cut into the graft material at the in-situ fenestration site.

17. A method of forming an in-situ fenestration, the method comprising:
advancing a cutting staple within a delivery catheter, the cutting staple including a cutting edge;
further advancing the cutting staple such that the cutting edge cuts into graft material at an in-situ fenestration site to form an in-situ fenestration; and
yet further advancing the cutting staple such that the cutting staple transitions into an anchored position in which the cutting staple pinches the graft material at a periphery of the in-situ fenestration.

18. The method of claim 17 further comprising removing the cut graft material from the in-situ fenestration.

19. The method of claim 18, wherein the removing step includes removing the cut graft material through a branch artery and the advancing step includes advancing a cutting staple within a delivery catheter situated within an aortic arch.

20. The method of claim 17, wherein the yet further advancing step including deforming the cutting staple as part of the transition into the anchored position.
